(19) United States
(12) Patent Application Publication
(10) Pub. No.: US 2011/0015191 A1
(43) Pub. Date: Jan. 20, 2011

(54) ORGANIC COMPOUNDS

(75) Inventors: Johannes Ludescher, Breitenbach (AT); Josef Wieser, Polling (AT); Arthur Pichler, Jenbach (AT); Ulrich Griesser, Axams (AT); Doris Braun, Dornbirn (AT)

Correspondence Address: Manelii Denison & Selter PLLC, 2000 M Street, 7th Floor, Washington DC, DC, 20036, US

(73) Assignee: SANDOZ AG, Basel (CH)

(21) Appl. No.: 12/528,483

(22) PCT Filed: Feb. 22, 2008
(86) PCT No.: PCT/EP2008/052192

§ 371 Date: Oct. 1, 2010

(30) Foreign Application Priority Data
Feb. 27, 2007 (EP) 07103150.4

(51) Int. Cl. A61K 31/5377; C07D 413/06; A61P 1/08

(52) U.S. Cl. 514/230.8; 544/132

(57) ABSTRACT

Novel polymorph form III of Aprepitant and a method for preparation of novel form III is disclosed. New processes for the preparation of Aprepitant form II are disclosed. The processes involve transformation of form III to form II by heating in decalin and the precipitation of form II from a solvent or solvent mixture by cooling and/or addition of addition of seed crystals or an anti solvent. Solid dispersions containing Aprepitant form II in a suitable carrier are disclosed. A process for the preparation of the solid dispersion by evaporation of a solution of Aprepitant and the carrier in a suitable solvent is disclosed. Stable Aprepitant form II is disclosed. Further pharmaceutical compositions containing Aprepitant form II, III or solid dispersions containing Aprepitant form II in a suitable carrier are disclosed. A methanol solvate of Aprepitant is disclosed.

### FIELD OF THE INVENTION

The present invention is concerned with the preparation of known physical forms of Aprepitant, certain novel physical forms of Aprepitant, the preparation of the novel physical forms of Aprepitant, solid dispersions of physical forms of Aprepitant and pharmaceutical compositions containing the physical forms of Aprepitant or the solid dispersion of the physical forms.

### BACKGROUND OF THE INVENTION

Aprepitant, 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine is a well known antagonist for the receptors of substance P and other tachykinin peptides. The neuropeptide receptors for substance P (neurokinin-1; NK-1) are distributed throughout the mammalian nervous system and are involved in regulating a number of biological processes including sensory perception of olfaction, vision, pain, movement control, vasodilatation etc. Substance P is a naturally occurring undecapeptide belonging to the tachykinin family of peptides, acting as e.g. vasodilator, depressant, stimulator of salvation etc.

Substance P antagonists are being investigated for neuropsychiatric diseases, including bipolar disorder, depression, anxiety mania and schizophrenia, as well as post herpetic neuralgia and pain. Substance P antagonists are also being investigated for the treatment of emesis. A therapeutic indication for Aprepitant is the prevention of nausea and vomiting associated with emetogenic cancer therapy.

Aprepitant is disclosed in PCT application No. WO 95/16679 A1 in example 75. It is known from the literature that Aprepitant exists in two polymorphic forms. U.S. Pat. No. 6,096,742 and U.S. Pat. No. 6,583,142 disclose the preparation of form I and form II of Aprepitant. Form I is reported to be the thermodynamically stable form. Further, the '742 patent discloses that form II converts to form I when heated to 230° C. under a nitrogen atmosphere. No information is given about the stability of form II at ordinary conditions. U.S. Pat. No. 6,096,742 discloses the preparation of form II of Aprepitant by recrystallization of Aprepitant obtained directly in the chemical synthesis. Form II of Aprepitant is characterized by powder XRD at 2-Theta angles of 12.6°±0.2°, 16.7°±0.2°, 17.1°±0.2°, 17.2°±0.2°, 18.3°±0.2°, 20.4°±0.2°, 20.7±0.2°, 21.1±0.2°, 22.9±0.2°, 23.9°±0.2° and 24.8°±0.2°. Form II may be easily distinguished from form I by interference free reflexes at angles 2-Theta of 12.6°±0.2°, 18.3°±0.2°, 20.7°±0.2°, 21.1°±0.2°, and 22.9°±0.2°.

Drugs of rather low solubility show enhanced solubility when they are present in thermodynamic less stable but kinetically stable forms. Such thermodynamic less stable forms may provide a greater aqueous concentration of the drug and thus a better bioavailability. Form II of Aprepitant has a solubility in a methanol/water mixture (2:1) of about 1.3±0.2 mg/ml compared to the solubility of 0.9±0.1 of form I.

### SUMMARY OF THE INVENTION

The present invention relates to novel form III of Aprepitant characterized by an X-ray powder diffraction pattern with peaks of about 6.78±0.2°, 7.44±0.2°, 11.86±0.2°, 12.63±0.2°, 17.1±0.2°, 18.32±0.2°, 18.7±0.2° 19.32±0.2°, 19.74±0.2°, 20.19±0.2°, 20.61±0.2° and 21.01±0.2° degrees two theta. More preferably, Form III of Aprepitant has substantially the same X-ray powder diffraction pattern as shown in FIG. 1.

The present invention further relates to Aprepitant methanol solvate characterized by an X-ray powder diffraction pattern with peaks of about 9.72±0.2°, 10.53±0.2°, 14.08±0.2°, 21.21±0.2° and 22.26±0.2° degrees two theta. More preferably, Aprepitant methanol solvate has substantially the same X-ray powder diffraction pattern as shown in FIG. 2.

The present invention provides a process for preparing form III of Aprepitant, comprising the steps of:

a) dissolving Aprepitant in tetrahydrofuran, dimethoxyethan or dioxan,
b) contacting the solution with C₅-C₁₀ aliphatic or alicyclic hydrocarbon to form a precipitate and
c) isolating the precipitate, which is the form III of Aprepitant.

In another aspect the invention provides a method for preparing essentially pure form II of Aprepitant where other crystalline Aprepitant forms are present at an amount of less than 5% that includes heating a suspension of form III in a solvent where form III is practically insoluble.

In yet another aspect the invention provides a process for preparing substantially pure form II of Aprepitant where Aprepitant form I is present at an amount of less than 40%, e.g. less than 30% of form I where substantially pure form II is crystallised from a solution of Aprepitant in a first solvent by addition of a second solvent, wherein the first solvent shows the better solubility for Aprepitant compared to the second solvent which shows lower solubility.

Another embodiment of the invention encompasses a method of preparing substantially pure form II of Aprepitant where crystallisation of substantially pure form II from a saturated solution of Aprepitant is induced by cooling the solution. Optionally the solution may be seeded and/or concentrated by evaporation.

In another embodiment the invention provides stable mixtures of substantially pure form II of Aprepitant containing less than 40%, e.g. less than 30% of Aprepitant form I.

In yet another embodiment the invention provides solid dispersions of substantially pure form II of Aprepitant in a suitable carrier.

In yet another embodiment the invention provides a process for the preparation of the solid dispersion of substantially pure form II of Aprepitant by mixing a solution of Aprepitant with the carrier and isolating the solid dispersion.

In yet another embodiment the invention relates to a pharmaceutical composition comprising form III of Aprepitant and relates also to a pharmaceutical composition comprising essentially or substantially pure form II of Aprepitant or a solid dispersion of substantially pure form II of Aprepitant.

### SHORT DESCRIPTION OF THE DRAWINGS

FIG. 1 shows the X-ray diffraction pattern of form III of Aprepitant.

FIG. 2 shows the X-ray diffraction pattern of the methanol solvate of Aprepitant.

FIG. 3 illustrates the X-ray diffraction pattern for essentially pure form II of Aprepitant.

FIG. 4 illustrates the X-ray diffraction pattern for substantially pure form II of Aprepitant having 10% of Form I.

FIG. 5 illustrates the X-ray diffraction pattern for substantially pure form II of Aprepitant having 20% of form I.

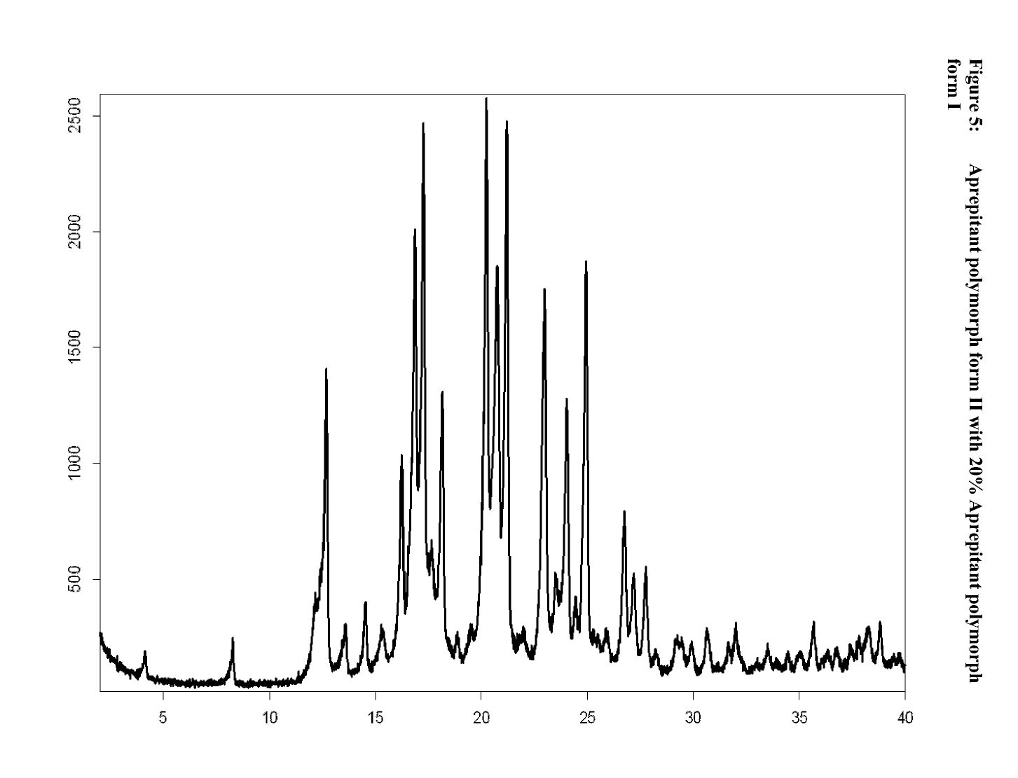

FIG. 6 illustrates the X-ray diffraction pattern for substantially pure form II of Aprepitant having 30% of Form I.

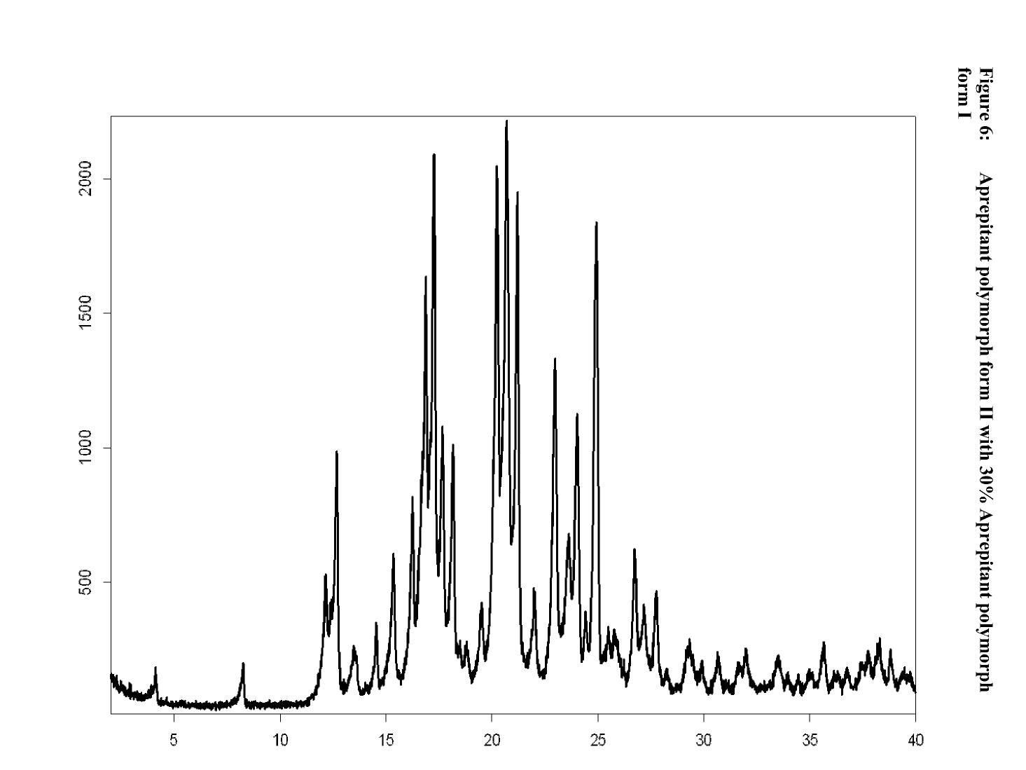

FIG. 7 shows the FTIR spectrum of form III of Aprepitant.

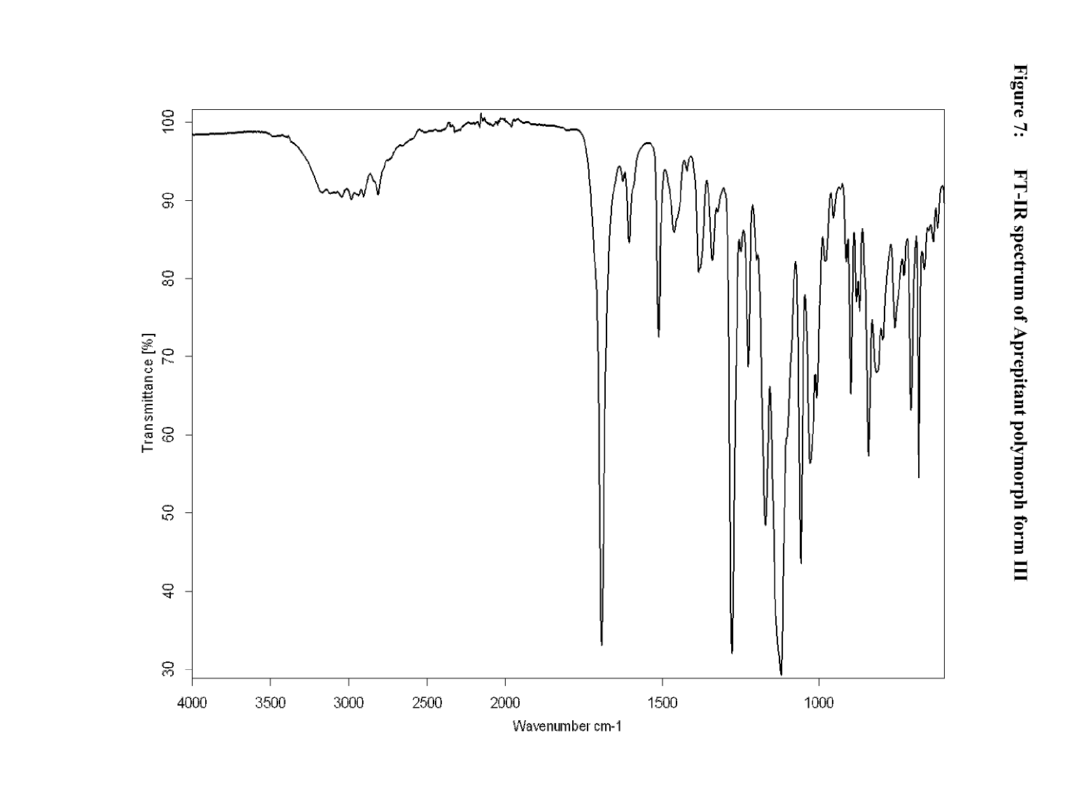

FIG. 8 illustrates the FTIR spectrum for essentially pure form II of Aprepitant.

FIG. 9 illustrates the FTIR spectra between 1170 and 1090 cm−1 of aprepitant polymorph mixtures containing 0, 10, 20, 30, 40 and 100% form I in form II.

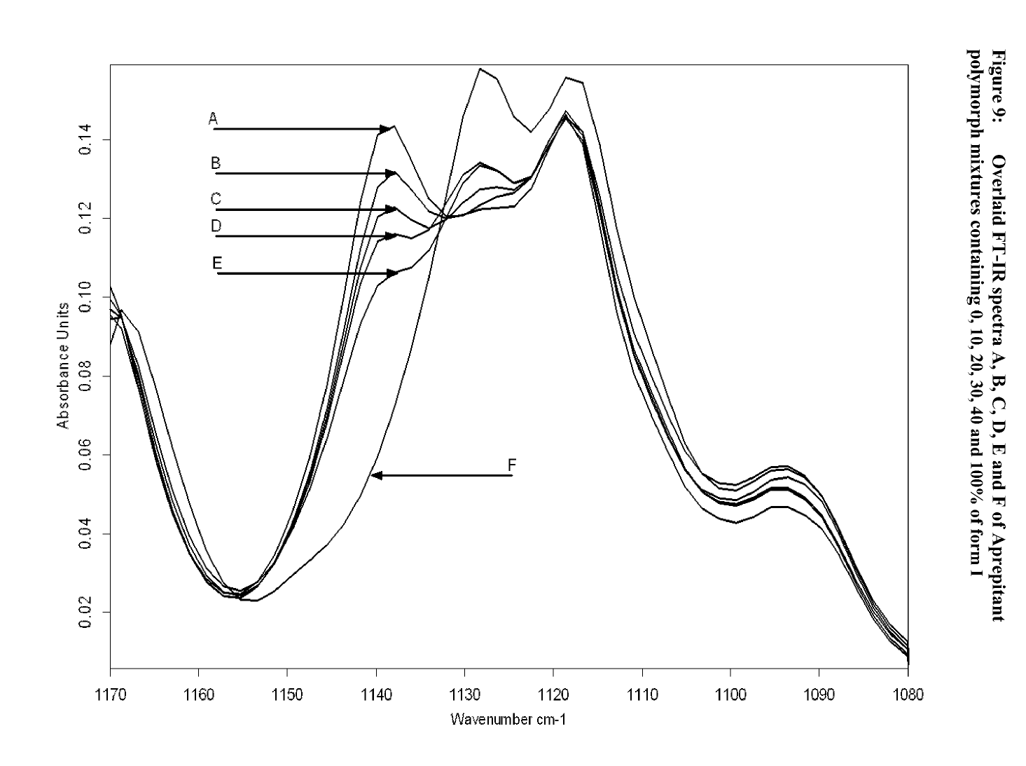

FIG. 10 shows the X-ray diffraction pattern of the solid dispersion of form II of Aprepitant and pentaerythritol.

FIG. 11 shows the X-ray diffraction pattern of the solid dispersion of form II of Aprepitant and PEG 6000.

FIG. 12 shows the X-ray diffraction pattern of the solid dispersion of form II of Aprepitant and sorbitol.

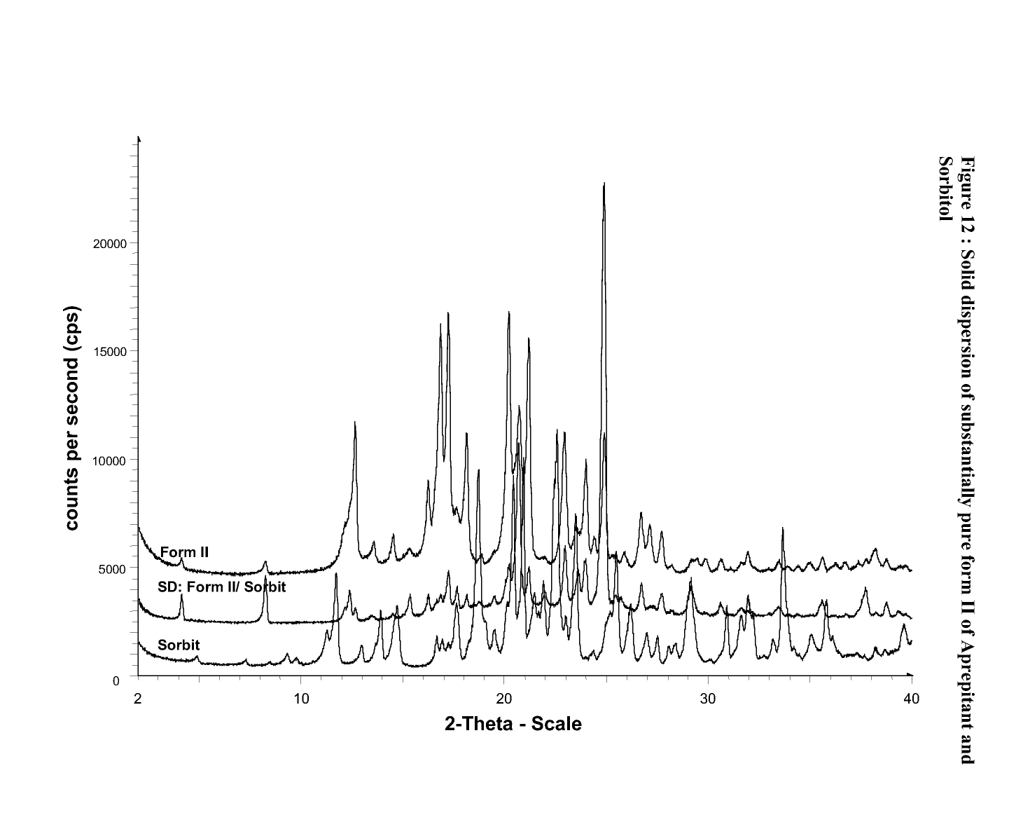

FIG. 13 shows the X-ray diffraction pattern of the solid dispersion of form II of Aprepitant and mannitol.

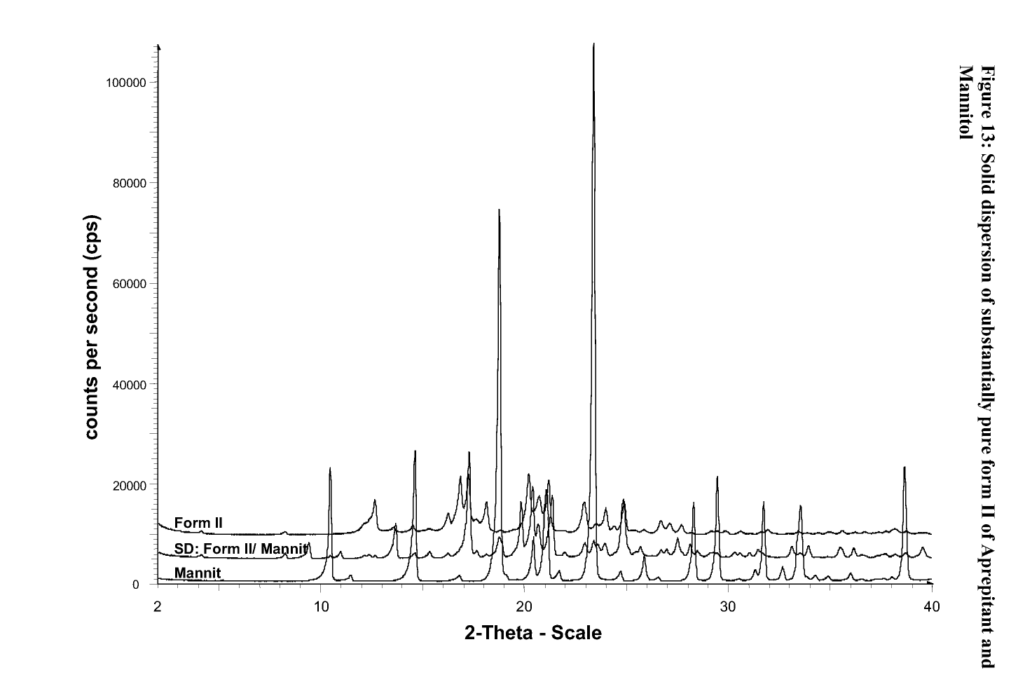

FIG. 14 shows the X-ray diffraction pattern of the solid dispersion of form II of Aprepitant and xylitol.

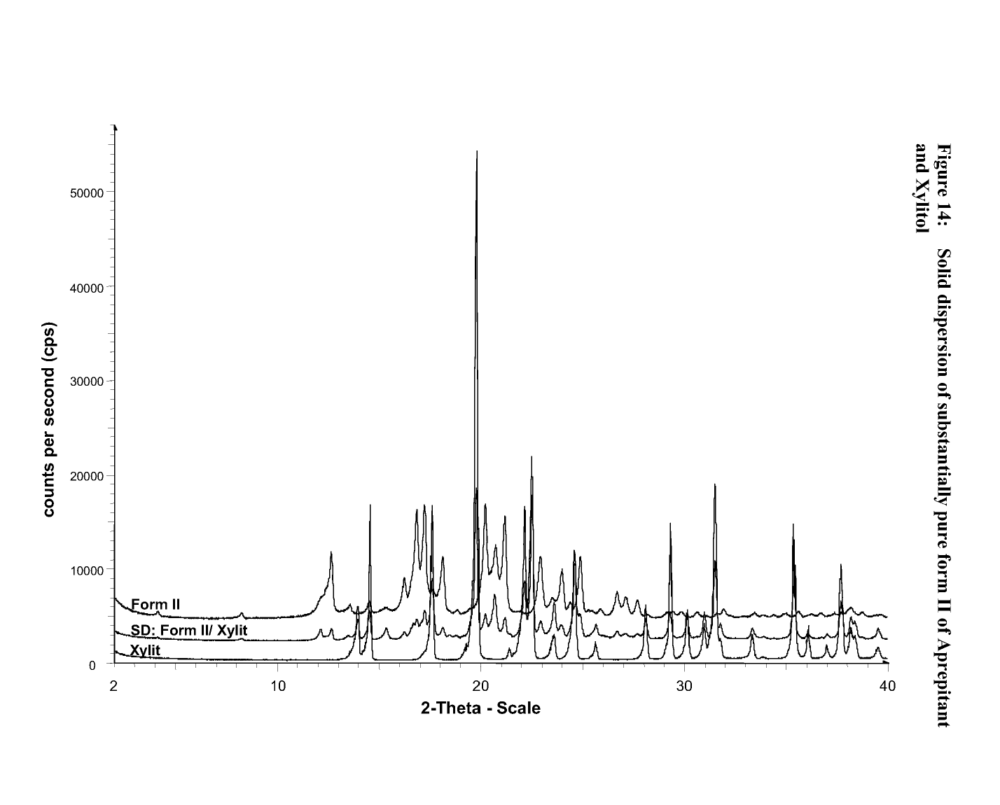

FIG. 15 shows the X-ray diffraction pattern of the solid dispersion of form II of Aprepitant and dextrose.

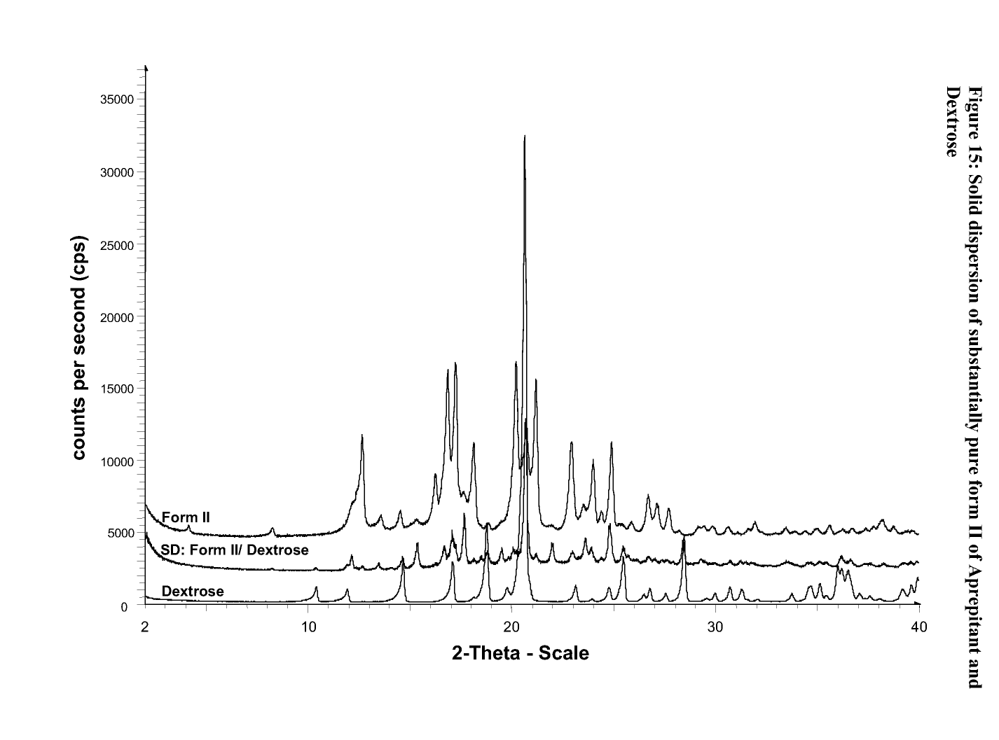

FIG. 16 shows the X-ray diffraction pattern of the solid dispersion of form II of Aprepitant and maltose.

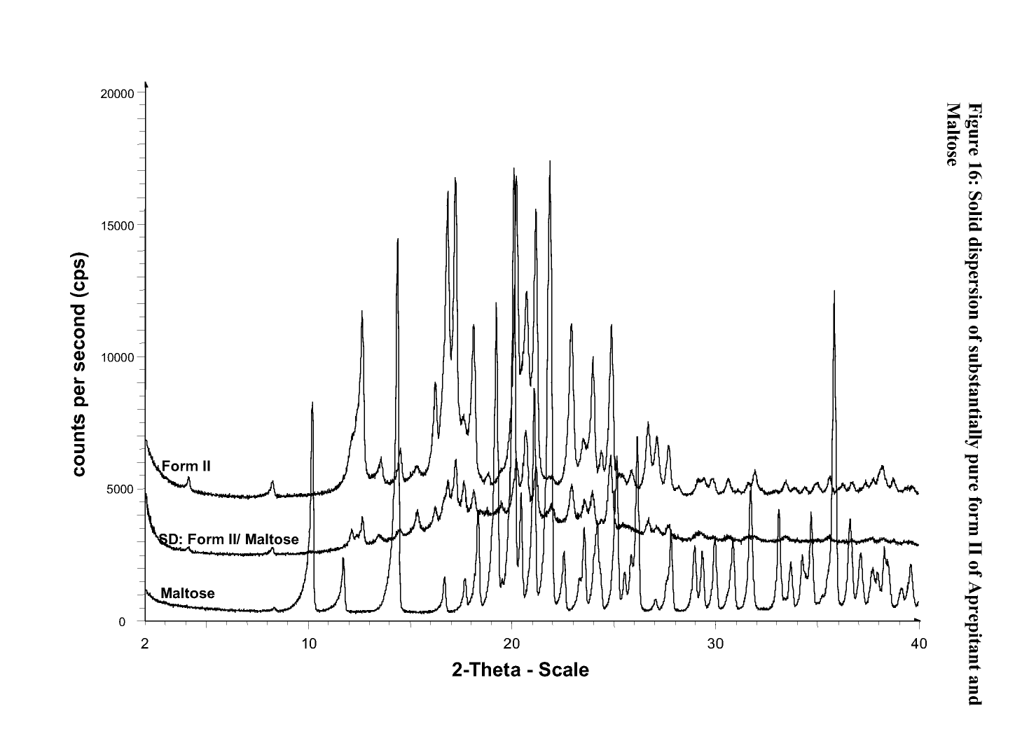

FIG. 17 shows the X-ray diffraction pattern of the solid dispersion of form II of Aprepitant and sucrose.

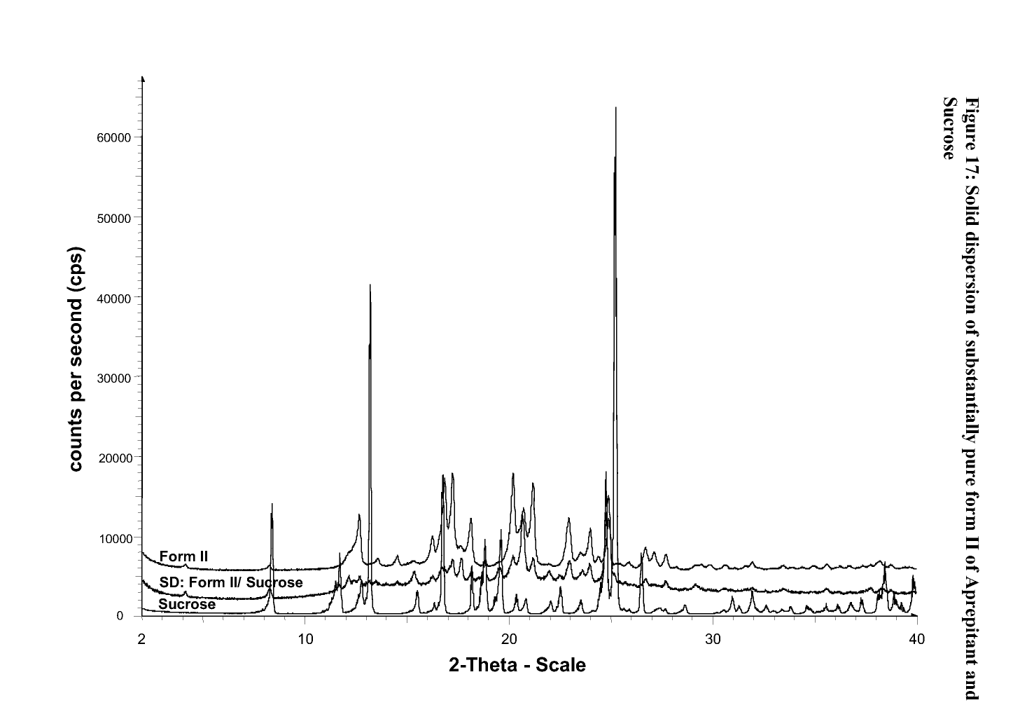

FIG. 18 shows the X-ray diffraction pattern of the solid dispersion of form II of Aprepitant and hydroxymethylpropylcellulose phthalate.

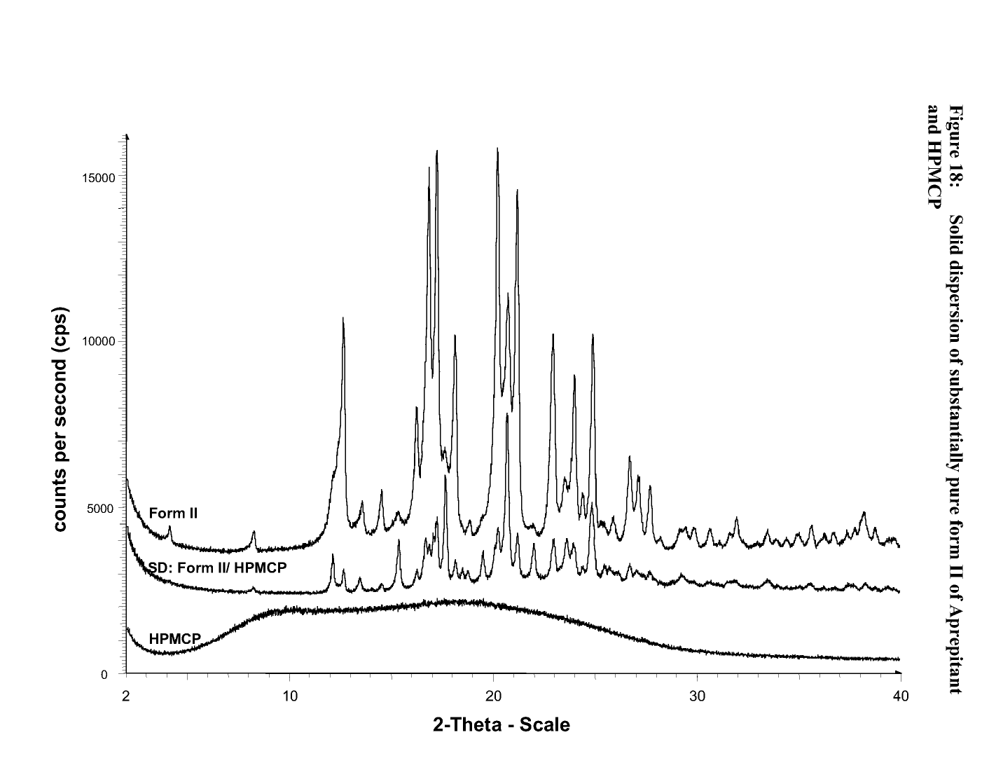

FIG. 19 shows the DSC of Aprepitant polymorph form III.

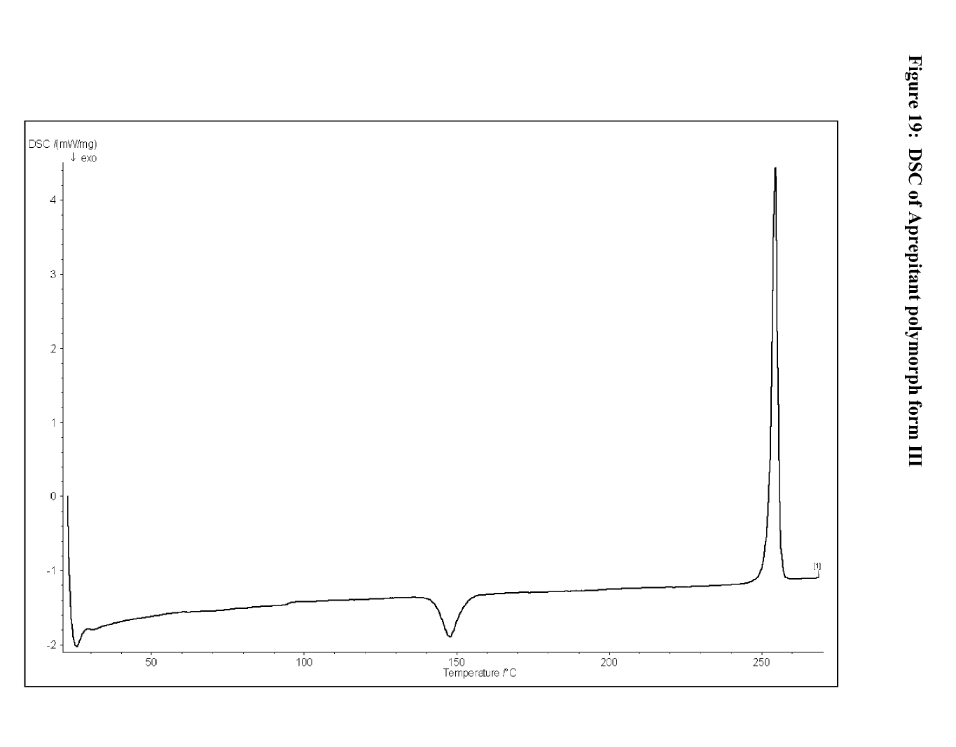

FIG. 20 shows the DSC/TGA of the methanol solvate of Aprepitant.

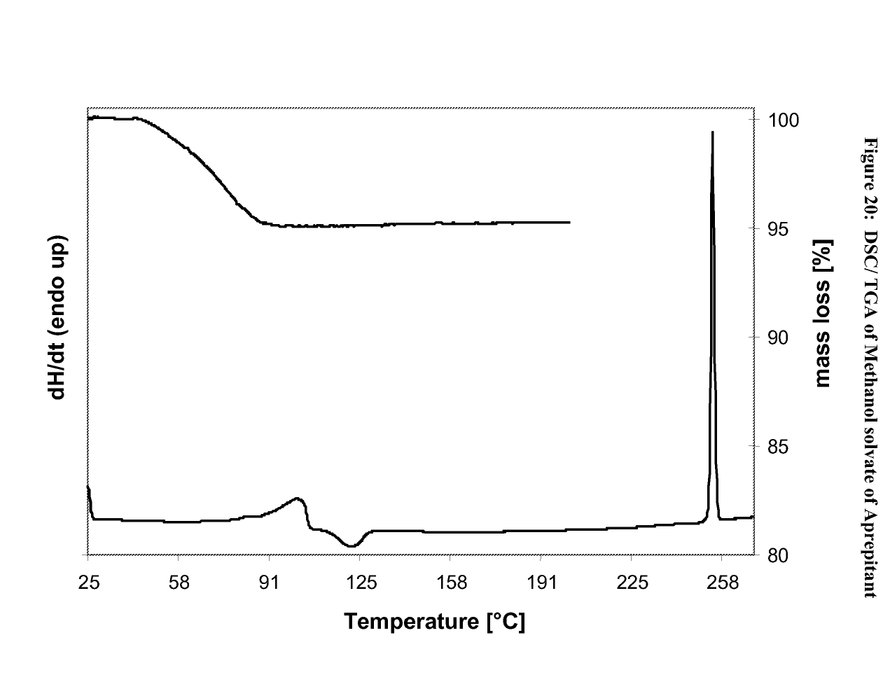

### DETAILED DESCRIPTION OF THE INVENTION

The names Aprepitant form III, form III of Aprepitant, form III and crystalline form III of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are used interchangeable in this application. All of these names are to be understood as the crystalline form III of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine.

The names Aprepitant form II, form II of Aprepitant, form II and crystalline form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are used interchangeable in this application. All of these names are to be understood as the crystalline form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine.

The names Aprepitant form I, form I of Aprepitant, form I and crystalline form I of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are used interchangeable in this application. All of these names are to be understood as the crystalline form I of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine.

Ambient temperature is to be understood as a temperature ranging from 18° C. to 30° C.

The statement: “C₁-C₆ alcohols” is to be understood as alcohols containing from one to six carbon atoms in the molecule.

The statement: “C₁-C₈ ketones” is to be understood as ketones containing from one to eight carbon atoms in the molecule.

The statement: “C₁-C₄ organic acids” is to be understood as monocarboxylic acids containing from one to four carbon atoms in the molecule.

The statement: “acetic acid C₁-C₄ alkyl esters” are to be understood as alkyl esters of acetic acid where the alkyl part contains from one to four carbon atoms.

The statement: “C₅-C₁₀ aliphatic or alicyclic hydrocarbon” are to be understood as non aromatic hydrocarbons such as n-hexane, n-heptane, cyclohexane or methylcyclohexane.

One embodiment of the invention encompasses a crystalline Aprepitant form, herein defined as form III, characterized by powder XRD at 2-Theta angles. The powder XRD shows characteristic peaks at angles 2-Theta(°) of about 6.78±0.2°, 7.44±0.2°, 11.86±0.2°, 12.63±0.2°, 17.1±0.2°, 8.32±0.2°, 18.7±0.2°, 19.32±0.2°, 19.74±0.2°, 20.19±0.2°, 20.61±0.2° and 21.01±0.2°.

Form III may be also characterized by DSC showing a broad exotherm in the range of about 140° C. to about 160° C. (heating rate 10° C./min) and a melting endotherm at about 253° C.

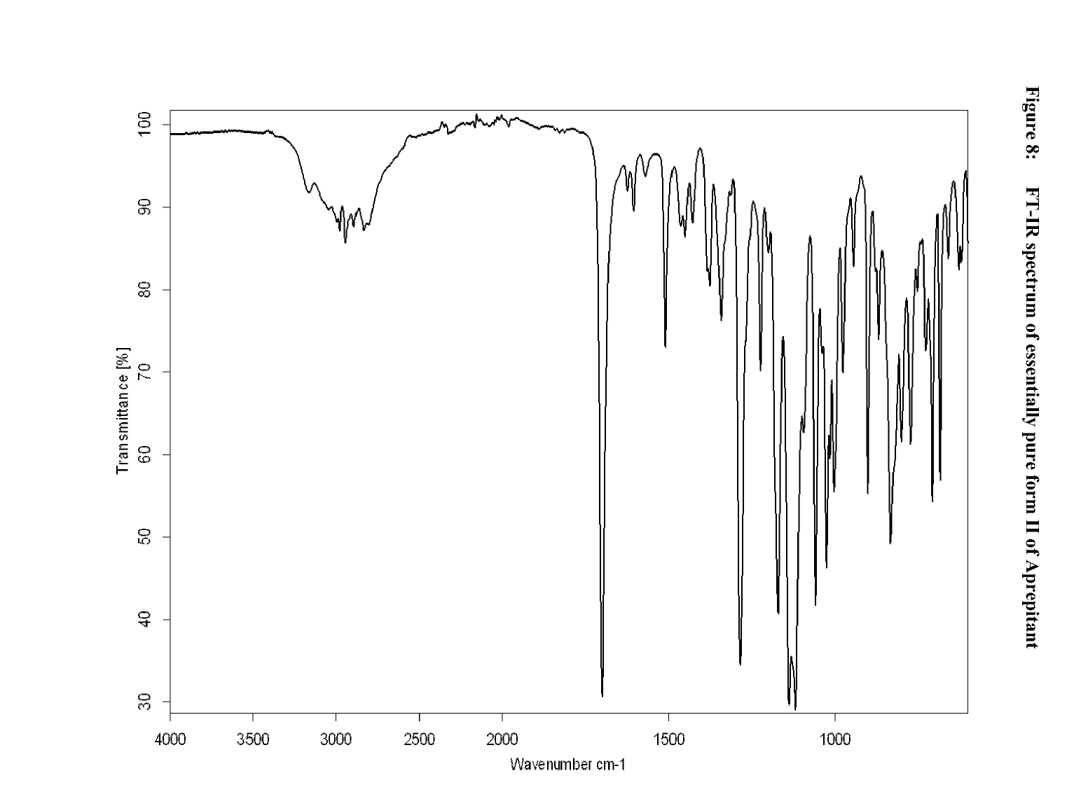

In addition form III may be also substantially identified by the FTIR spectrum of FIG. 8. Characteristic bands are present at 3043, 2905, 1693, 1170, 1121 and 815 cm⁻¹.

The invention also encompasses a method of preparing form III comprising the steps of:

a) dissolving Aprepitant in tetrahydrofuran, dimethoxyethan or dioxan,
b) contacting the solution with C₅-C₁₀ aliphatic or alicyclic hydrocarbon to form a precipitate and
c) isolating the precipitate, which is the form III of Aprepitant.

The required solution of Aprepitant in step a) of this process may be obtained by dissolving Aprepitant at room temperature or by heating Aprepitant in the chosen solvent. In general, the concentration of Aprepitant in the solvent is the maximum concentration; i.e., the saturation concentration. A particularly useful solvent for use in step a) is tetrahydrofuran. Typically the concentration using tetrahydrofuran as solvent is about 200 mg/ml. The solution is preferably passed through a filter so that the solution is free of any contamination by any other polymorph. In step b) the solution of Aprepitant in the solvent is combined with a C₅-C₁₀ aliphatic or alicyclic hydrocarbon as antisolvent. Preferably the Aprepitant solution is added to the hydrocarbon with stirring. Generally, the hydrocarbon is of ambient temperature or less. The ratio of solvent and hydrocarbon in the precipitation step is between 1:9 and 1:30 or higher. The isolation of the precipitate may be carried out by any conventional method. In general, the solid material is recovered from the liquid portion such as by filtration or centrifugation, optionally washed with the hydrocarbon and dried. The drying can be conducted in vacuo with or without applying heat. The drying temperature preferably does not exceed 40° C.

The process for the preparation of Aprepitant form II that is disclosed in U.S. Pat. No. 6,096,742 involves an isolation step where Aprepitant form II is crystallized from a solution of Aprepitant in methanol by dropwise addition of water. The ratio of methanol and water in the precipitation step is 2.37:1 (v/v).

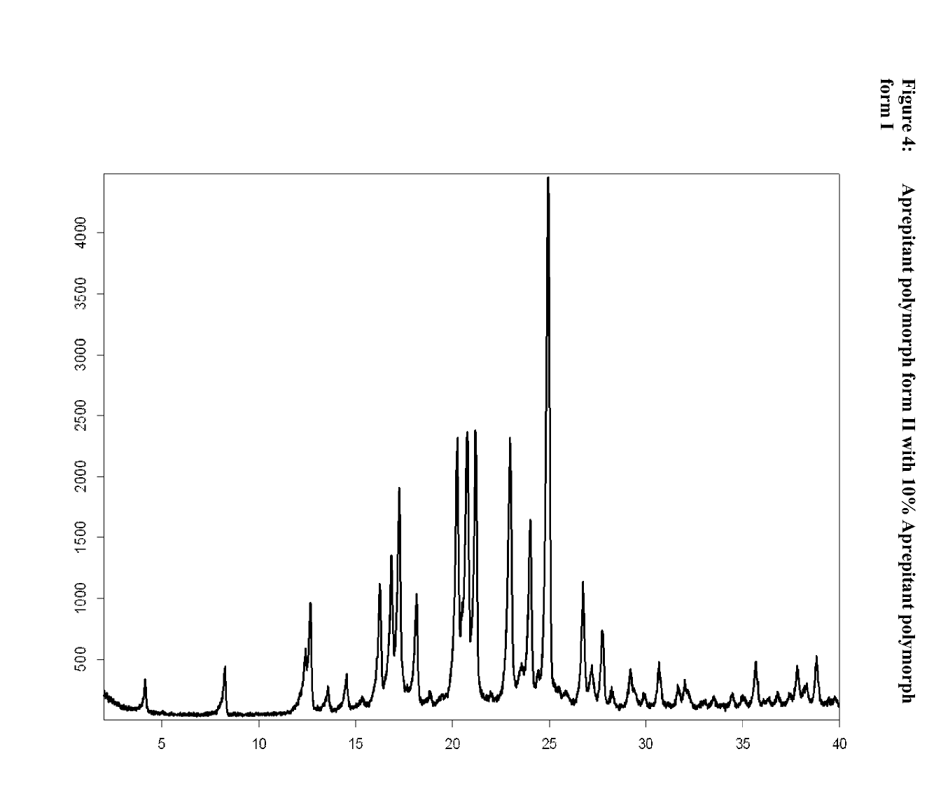

In our hands pure form II of Aprepitant may not be obtained by a crystallization procedure. Reproducing example 20 of WO 99/01444 resulted in an approximately 1:1 mixture of form II and form I (see comparative example 1). Similar conditions in crystallization from a 1:1 mixture methanol and water resulted in a mixture containing about 76% of form I and only about 24% of form II. The XRD-pattern in U.S. Pat. No. 6,096,742 does not represent pure form II of Aprepitant when compared with FIG. 4 of the present application.

Thus a drawback of the '742 process is that it produces a mixture of polymorphic forms and there is need for an improvement in the ratio of form II to form I. There is also a need for a reliable process for the improved ratio of form II to form I of Aprepitant.

In one embodiment the invention provides a process for the preparation of substantially pure form II of Aprepitant with an amount of less than 40%, or less than 30% of Aprepitant form I where said mixture II is crystallised from a solution of Aprepitant in a first solvent by addition of a second solvent in an amount of at least 2 volumes of the second solvent, wherein the first solvent shows the better solubility for Aprepitant compared to the second solvent which shows lower solubility.

In another embodiment the invention provides a process for the preparation of substantially pure form II containing less than 40%, e.g. less than 30% of form I where crystallisation is induced by cooling the solution. Optionally the solution may be seeded and/or concentrated by evaporation.

In one embodiment substantially pure form II containing less than 40% of form I is prepared by providing a solution of Aprepitant in a first solvent followed by mixing the solution with a second solvent wherein Aprepitant has a lower solubility in a ratio of first solvent to the second solvent of at least 1:2. A preferred ratio is from about 1:2 to a ratio of about 1:5.

The temperature of the solution may vary from about ambient temperature to the boiling point of the first solvent, preferably from about ambient temperature to about 100° C. The most preferred temperature is ambient temperature. Cooling may be applied after the addition of the second solvent if appropriate. The formed crystals may be isolated by filtration, centrifugation or by decanting the solvent. The isolated crystals may be dried by conventional drying procedures, e.g. by air drying, drying under a flow of nitrogen or vacuum drying.

The mixing of the first solvent and antisolvent is preferably performed rapidly in a way to achieve a high degree of supersaturation.

Increasing the ratio of the second solvent to the first solvent from approximately 2:1 to 3:1 or more, substantially pure Form II of Aprepitant containing less than about 40% of form I of Aprepitant may be produced in a reproducible manner by presence or absence of seeds.

Thus the present invention encompasses the reproducible preparation of substantially pure form II of Aprepitant containing less than about 30% or less than about 40% of Aprepitant form I by mixing a solution of Aprepitant in a first solvent with a second solvent in a ratio of about at least 1:2. Preferably the second solvent is admixed with the solution of Aprepitant. Preferably the mixing is performed quickly.

The first solvent may be selected from C₁-C₈ ketones such as acetone or methyl ethyl ketone, halogenated alkanes such as chloroform or dichloromethane, acetic acid C₁-C₄ alkyl esters such as ethyl acetate, C₁-C₆ alcohols such as 1-propanol, ethers like dioxane and tetrahydrofuran. The second solvent may be selected from water or an alkane, e.g. heptane.

Preferred combinations of first solvents and second solvents are the combinations of acetone, 1-propanole and tetrahydrofuran with the second solvent water, preferred combinations are ethyl acetate, acetone, chloroform, dichloromethane, dioxane or methyl ethyl ketone with the second solvent being heptane.

Another embodiment encompasses the crystallization of substantially pure form II of Aprepitant containing less than about 40%, e.g. less than about 30% of form I of Aprepitant from a saturated solution of Aprepitant in a solvent or solvent mixture. A saturated solution is prepared by the dissolution of Aprepitant in a solvent or solvent mixture where the solvent or solvent mixture is held at a temperature from about ambient temperature to the boiling point of the solution. Crystallization is induced by cooling of the saturated solution. Seeds of form II may be added as part of the crystallization procedure. Crystallisation may be effected by cooling the solution or suspension formed to a temperature from about ambient temperature to −50° C., preferably to a temperature of about −20° C. to about 10° C., even more preferably to a temperature of about −20° C. to about 0° C. Cooling may be performed slowly, e.g. within several hours or fast. Fast cooling is to be understood as a cooling procedure where the solution is cooled to the desired temperature within approximately 1 to 120 min. In a preferred cooling procedure the solution is cooled to the desired temperature within 30 min. Slow cooling is to be understood as a cooling procedure where the solution is cooled to the desired temperature within approximately 2 to 24 hours. Optionally part of the solvent may be removed prior to or during the crystallization step to obtain a saturated solution. The formed crystals may be isolated by filtration, centrifugation or by decanting the solvent. Drying may be performed by conventional drying procedures, e.g. by air drying, drying under a flow of nitrogen or vacuum drying.

Suitable solvents for use in this embodiment may be ethers, C₁-C₆ alcohols or aromatic hydrocarbons. Preferred solvent are 2-propanol, 1-butanol, dioxan, tetrahydrofuran, toluene or xylene.

Suitable solvent mixtures for use in this embodiment mixtures of C₁-C₆ alcohols and water, mixtures of aromatic hydrocarbons and C₁-C₈ ketones, mixtures of C₁-C₃ ketones and water, mixtures of C₁-C₄ organic acids and water, mixtures of amides and water and mixtures of nitroalkanes and water.

Preferred solvent mixtures are mixtures of acetone and water, methanol, ethanol, 1-propanol and 2-propanol and water, xylene and acetone, acetic acid and water, N,N-dimethylformamide and water and nitromethane and water. The amount of water present in the solvent mixture is preferably about 2 volumes of the organic solvent used.

It is known from the literature that Aprepitant form I is the thermodynamically stable form, and that Aprepitant form II converts into form I at elevated temperature. No informations about the stability of mixtures of Aprepitant form II and form I at ordinary temperatures are found in the literature.

It has now surprisingly been found, that mixtures of Aprepitant form II and Aprepitant form I prepared by the procedures described above are stable and do not convert into form I even at prolonged storage.

Thus, an embodiment of the invention are stable mixtures of form II and form I of Aprepitant. The mixture is stable characterised in that it does not convert to enriched form I when stored e.g. at about 0% rel. humidity at ambient temperature, or at about 0% rel. humidity at 60° C. or at about 100% rel. humidity at 60° C. for at least 2 months.

A particular embodiment of the invention encompasses a method of preparing essentially pure form II. As used herein, the term “essentially pure” refers to form II having no more than 5% of other crystalline forms of Aprepitant. Whereas recrystallisation is not a reliable method for the preparation of form II in essentially pure form we have surprisingly found that form III of Aprepitant may be used for the preparation of essentially pure form II, which can consistently be prepared by heating a suspension of form III in a solvent where form III is practically insoluble. Preferably, form III is heated in decalin at about 100° C. to about 120° C. for about 5 to about 50 minutes. Small increases in temperature may have a significant effect on the time required for the formation of form II.

Solid dispersions play an important role in pharmaceutical technology. Solid dispersions may enhance physical/respectively chemical stability of drugs and may contribute significantly to the bioavailability of the drug.

It would therefore be desirable if solid dispersions containing Aprepitant could be obtained.

Another embodiment of the invention is a solid dispersion comprising substantially pure form II of Aprepitant in a suitable carrier. The ratio of Aprepitant form II and form I: carrier may be in the range of about 1:1 to about 1:10, preferably in the range of about 1:1 to about 1:3. The carrier may be selected from macrogols, succinic acid, urea, pectin, desoxychloic acid, galactomannan, urethane, methylcellulose, hydroxypropyl cellulose, hydroxypropylmethylcellulose phthalate, polyethylenglycol, poloxamers, polyacrylates, polymethylacrylates, hydroxyalkylxanthine, dextrose, sucrose, galactose, maltose, xylitol, cyclodextrin, mannitol and sorbitol. Preferred carriers are polyethylenglycol, e.g. PEG 6000, maltose, sucrose, hydroxypropylmethyl cellulose or hydroxypropylmethyl cellulose phthalate.

Another embodiment of the invention is a process for the preparation of a solid dispersion comprising substantially pure form II in a suitable carrier. Solid dispersions of substantially pure form II of Aprepitant may be prepared by evaporation of a solution of a mixture of Aprepitant and a carrier, or by evaporation of a suspension of a carrier in a solution of Aprepitant. The solvent evaporation may be performed by using reduced pressure, lyophilization or spray drying.

Suitable solvents for the use in the preparation of solid dispersions of mixtures of Aprepitant form II and form I are alcohols such as methanol or ethanol, ketones such as acetone, or mixtures of one or more alcohols and one or more ketones, optionally in the presence of water. Suitable carriers for the use in the preparation of solid dispersions of substantially pure form II of Aprepitant may be selected from macrogols, succinic acid, urea, pectin, desoxychloic acid, galactomannan, urethane, methylcellulose, hydroxypropylcellulose, hydroxypropylmethylcellulosephthalate, polyethylenglycol, poloxamers, polyacrylates, polymethylacrylates, hydroxyalkylxanthine, dextrose, sucrose, galactose, maltose, xylitol, cyclodextrin, mannitol and sorbitol. Preferred carriers are polyethylenglycol, e.g. PEG 6000, maltose, sucrose, hydroxypropylmethylcellulose or hydroxypropylmethylcellulose phthalate.

In another embodiment the invention relates to a novel methanol solvate of Aprepitant. The methanol solvate is useful for the purification of Aprepitant. The methanol solvate shows nice purification efficacy and converts to form I of Aprepitant on drying. The methanol solvate of Aprepitant shows desolvation and a melting process from about 70° C. to 105° C. at a heat rate of about 10K/min. The powder XRD shows characteristic peaks at angles 2-Theta(°) of about 9.72±0.2°, 10.53±0.2°, 14.08±0.2°, 21.21±0.2° and 22.26±0.2°.

A process for the preparation of the methanol solvate of Aprepitant is another embodiment of the invention. The methanol solvate may be prepared by dissolving Aprepitant in methanol and cooling the solution to about 0° C. to about −50° C., preferably to a temperature of about −10° C. to about −20° C.

The invention further relates to a pharmaceutical composition comprising form III or essentially pure form II or substantially pure form II of Aprepitant containing less than about 40% of Aprepitant form I, e.g. less than 30% of form I, or a solid dispersion of substantially pure form II.

The pharmaceutical compositions may comprise form III or essentially pure form II or substantially pure form II of Aprepitant containing less than about 40% of Aprepitant form I, e.g. less than 30% of form I or a solid dispersion of substantially pure form II that are made with the processes described above.

Preferred pharmaceutical compositions comprise substantially pure form II of Aprepitant containing less than about 40% of Aprepitant form I, e.g. less than 30% of form I or a solid dispersion thereof are oral dosage forms such as tablets, capsules, powder for oral suspensions, pills and granules.

The preferred compositions can be prepared by mixing substantially pure form II of Aprepitant containing less than about 40% of Aprepitant form I, e.g. less than 30% of form I or a solid dispersion thereof with pharmaceutically inert inorganic and/or organic excipients or additives and prepared into a desired dosage form.

Suitable excipients and additives include for example fillers, disintegrants, binders, lubricants, wetting agents, stabilizers, emulsifiers, preservatives, sweeteners, colorants, flavourings, aromatizers, thickening agents, diluents, buffer substances, solvents, solubilizers, agents for achieving a depot effect, salts for changing the osmotic pressure, coating agents and/or antioxidants.

Examples of suitable fillers include, but are not restricted to agents such as microcrystalline cellulose, lactose, sugars, starches, modified starch, mannitol, sorbitol and other polyols, dextrin, dextran and maltodextrin, calcium carbonate, calcium phosphate and/or hydrogen phosphate, sulphate.

Suitable binders include, for example, lactose, starches, modified starch, dextrin, dextran and maltodextrin, microcrystalline cellulose, sugars, polyethylene glycols, hydroxypropyl cellulose, hydroxypropyl methylcellulose, ethylcellulose, hydroxyethyl cellulose, methylcellulose, carboxymethyl cellulose, gelatine, acacia gum, tragacanth, polyvinylpyrrolidone, copolyvidone, and/or sodium alginate.

Suitable disintegrating agents comprises cross-carmellose sodium, cross-linked polyvinylpyrrolidone, cross-linked carboxymethyl starch, starches, sodium starch glycolate microcrystalline cellulose, magnesium aluminium silicate and/or polyacrylin potassium.

Suitable lubricants according to the invention comprise agents such as magnesium stearate, calcium stearate, zinc stearate, calcium behenate, sodium stearyl fumarate, talc, magnesium trisilicate, stearic acid, palmitic acid, carnauba wax and/or colloidal silicon dioxide.

Furthermore, if required any, the composition may also include surfactants and other conventional components for solid, pharmaceutical compositions such as colouring agents, lakes, flavours and/or adsorbents.

For example Aprepitant according to the invention may be formulated as a capsule comprising from 80 mg to 125 mg Aprepitant and further sucrose, microcrystalline cellulose, hydroxypropyl cellulose and sodium lauryl sulphate. The capsule shell may contain e.g. gelatine, iron oxide, titanium dioxide and silicon dioxide.

The invention is now further described by examples, which are provided for illustrative purposes and are not intended to be limiting for the invention in any way.

The amount of form I and form II in a mixture of form I and II, e.g. in substantially pure form II of Aprepitant containing less than about 40% of Aprepitant form I, e.g. less than 30% of form I can be determined using standard solid state analytical procedures such as X-ray powder diffractometry including peak-profile fitting and infrared spectroscopy including infrared spectroscopy with second derivative processing as described by Roy Helmy et al., Analytical Chemistry 2003, 75, 605-611.

Powder diffraction measurements for the quantification of form I in form II of Aprepitant were performed on a STADIP MP laboratory powder diffractometer in a transmission geometry with a 5° position sensitive detector using Cu Kal radiation, 6 mm axial beam height and 2 mm sample diameter. A diffraction angle range of 5°-45° was measured with total measuring time of 3 h and 45 min. The measurements were analysed by the Riethfeld method using the Fullprof.2K version 3.40. Structures of form II and form I from single crystal data have been used for the calculation of the intensities of the reflections. Isotropic Debey-waller fractions of B=2 were applied for each phase in addition to the anisotropic values from the single crystal refinement.

The atomic positions of form II were refined, using bond distance and angle restrains taken from the single crystal structure, atomic positions of form I were kept fixed. Further refinement parameters were lattice parameters of each phase, scale factors, isotropic particle size and microstrain broadening with common values for both phases, 18 background positions.

### EXAMPLES

### Comparative Example 1 according to WO 99/01444

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

0.133 g of Aprepitant is dissolved in 1.7 ml boiling methanol. The solution is then cooled to ambient temperature. To the solution is then added drop wise 0.7 ml of H₂O. The suspension is then stirred for 2 hours and the precipitate is isolated by filtration and dried.

Polymorphic purity approximately 50% form 1 and 50% form II (analysis by XRPD and FTIR)

### Comparative Example 2

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

0.1 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 2 ml of methanol at ambient temperature. 1 ml of water is added with stirring in 1 portion and a precipitate is formed.

Polymorphic purity approximately 25% form II and 75% form I (analysis by XRPD)

### Example 1

### Preparation of Form III of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

1 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 5 ml of tetrahydrofuran at ambient temperature. The solution is filtered to remove any undissolved material and then added to 150 ml n-hexane under stirring. After stirring under ice cooling for 30 min the precipitated solid is filtered and washed with n-hexane The resulting solid is dried under vacuum at room temperature to obtain aprepitant form III in 87% yield.

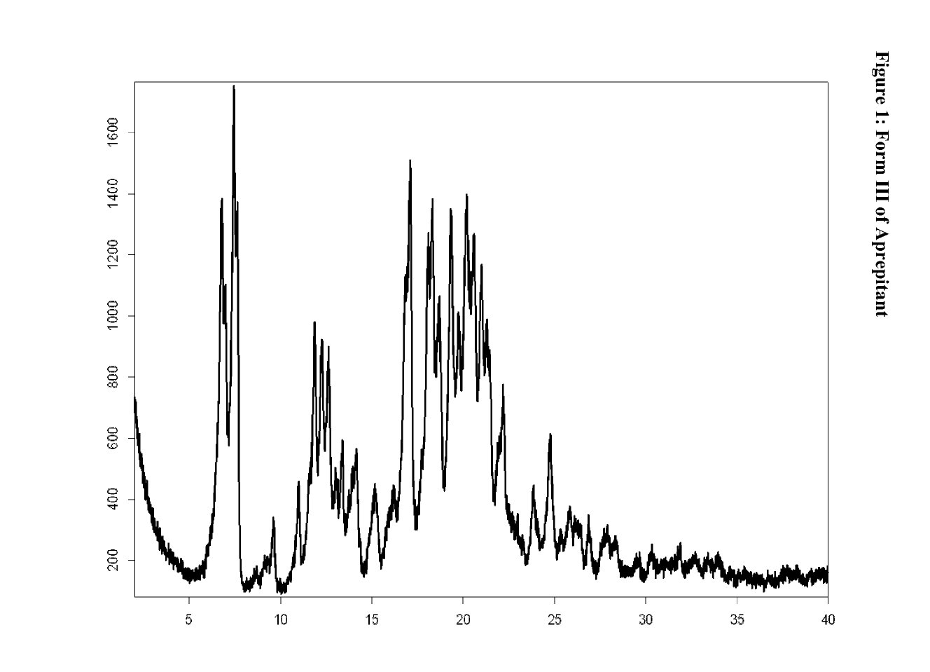

The XRPD pattern of form III of aprepitant with characteristic XRPD angles and relative intensities is shown in table 1 and FIG. 1.

TABLE 1
X-Ray Powder Diffraction (XRPD) pattern of
form III of aprepitant. Values: characteristic
XRPD angles (in degrees 2-theta) and relative
intensities (in %)

| 2-Theta | Rel. Intensity (%) |
|---|---|
| 6.78 | 78 |
| 7.44 | 100 |
| 9.61 | 19 |
| 11.01 | 26 |
| 11.86 | 55 |
| 12.25 | 52 |
| 12.63 | 51 |
| 13.37 | 33 |
| 14.14 | 32 |
| 15.18 | 25 |
| 17.1 | 85 |
| 18.32 | 78 |
| 18.7 | 60 |
| 19.32 | 76 |
| 19.74 | 57 |
| 20.19 | 79 |
| 20.61 | 71 |
| 21.01 | 66 |
| 21.32 | 55 |
| 22.19 | 44 |
| 23.86 | 25 |
| 24.76 | 35 |

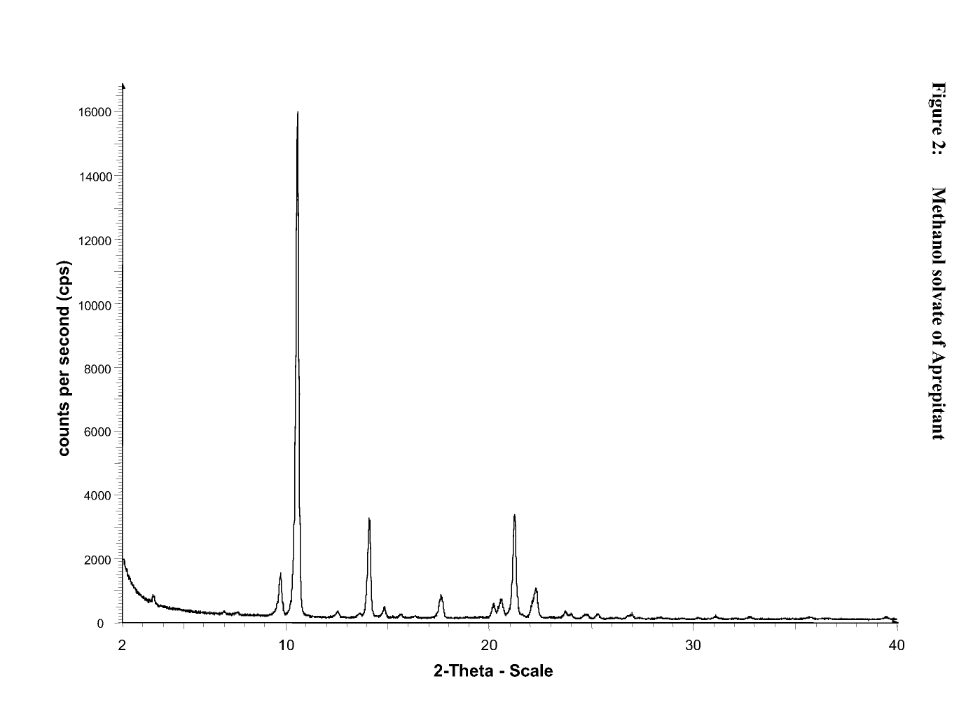

The form III of aprepitant obtained above has an infrared spectrum which is substantially identical to the IR spectrum shown in FIG. 2. Specifically, it has clear infrared absorption bands at 3043, 2905, 1693, 1170, 1121 and 815 cm⁻¹.

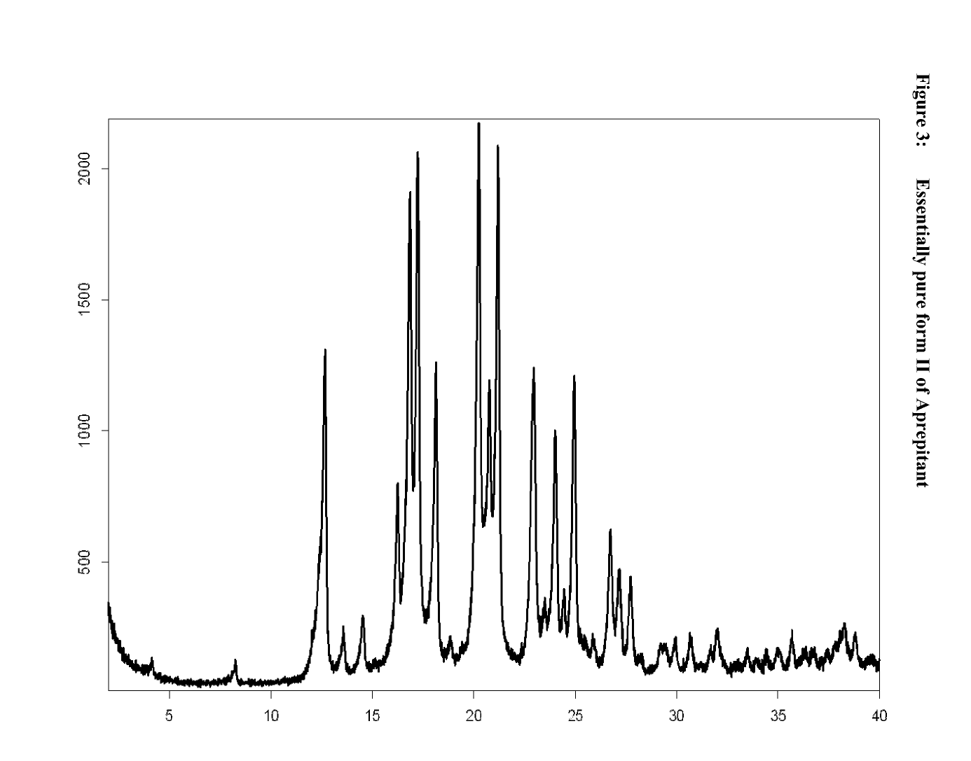

Form III of aprepitant shows a typical DSC curve at a heating rate of 10° K/min. A typical thermogram of aprepitant is shown in FIG. 3. It can be seen that form III of aprepitant shows a broad exotherm in the range of about 140° C. to about 160° C. and a melting endotherm at about 253° C.

### Example 2

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

0.1 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 2 ml of methanol at ambient temperature. Seeds of Aprepitant are added followed by 1 ml of water in one portion with stirring at ambient temperature. The precipitate is isolated by filtration and analysed by PXRD.

Polymorphic purity approximately 35% form 1 and 65% form II (analysis by XRPD)

### Example 3

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

0.095 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 2 ml of methanol at ambient temperature. Seeds of Aprepitant form II are added followed by 4 ml of water in 1 portion and a precipitate is formed.

Polymorphic purity approximately 30% form 1 and 70% form II (analysis by XRPD)

### Example 4

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

0.11 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 2 ml of methanol at ambient temperature. 4 ml of water are added in 1 portion and a precipitate is formed.

The crystals are isolated by filtration.

Polymorphic purity approximately 30% form 1 and 70% form II (analysis by XRPD)

### Example 5

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

0.11 g of 0.11 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 2 ml of methanol at ambient temperature. The solution is cooled in an ice bath and 4 ml of water are added in 1 portion. The crystals formed are isolated by filtration, dried in vacuo.

Polymorphic purity approximately 30% form 1 and 70% form II (analysis by FTIR)

### Example 6

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

1.2 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 20 ml of acetone at ambient temperature. After the addition of 40 ml water in a constant flow, the title compound crystallizes as form II. The suspension is filtered, and the isolated crystals are dried in vacuo at approximately 20 mbar.

### Yield: 1.13 g

Polymorphic purity approximately 70% form II (analysis by FTIR)

In a similar manner crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine form is performed by dissolving 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine at ambient temperature in a first solvent at ambient temperature and adding twice the volume of a second solvent. The experiments are summarized in table 2.

TABLE 2

| | Second | Form II |
|---|---|---|
| Solvent | solvent | % FTIR |
| Acetone | Water | >=70 |
| | Heptane | >=70 |
| Dichloromethane | Heptane | >=70 |
| Dioxane | Heptane | >=70 |
| Ethylacetate | Heptane | >=70 |
| MEK | Heptane | >=70 |
| 1-propanol | Water | >=70 |
| Tetrahydrofuran | Water | >=70 |
| | Hexane | >=70 |
| | Xylene | >=70 |

### Example 7

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

1.2 g of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in a 1:2 mixture of ethanol/water (27 ml ethanol, 54 ml water). The solution is stirred under reflux for 30 minutes at 98° C. (water bath temperature), followed by cooling to room temperature within 4 hours to give crystalline Aprepitant. The suspension is filtered, and the isolated crystals are dried in vacuo at approximately 20 mbar.

### Yield 1.01 g.

FTIR shows the presence of approximately 70% of form II

### Example 8

### Crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

1.4 g (1.3651 g) of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in a 1:2 mixture of acetic acid in water (10 ml acetic acid, 20 ml water) under heating. The solution is then stirred under reflux and reduced pressure (50 mbar) at 60° C. After the addition of seed crystals of form II the solution is fast cooled in an ice bath to give crystalline Aprepitant.

The suspension is filtered, and the isolated crystals are dried in vacuo at approximately 20 mbar.

### Yield 1.08 g.

FTIR shows the presence of approximately 60% of form II

In a similar manner to example 7 and example 8 crystallization of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine form II is performed by dissolving 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine at elevated temperature followed by cooling of the solution. The experiments are summarized in table 3 and 4.

TABLE 3

| | | Form II |
|---|---|---|
| Solvent | Method | % (FTIR) |
| Acetic acid + H₂O | fast cooling | >=60 |
| Acetic acid + H₂O | Slow cooling | >=60 |
| Acetone + H₂O | slow cooling | >=60 |
| 2-Propanol | Fast cooling | >=60 |
| 2-Propanol + H₂O | Fast cooling | >=60 |
| MeOH + H₂O | Slow cooling | >=60 |
| THF | Fast cooling | >=60 |
| Toluene | Fast cooling | >=60 |

Fast cooling: fast cooling of a hot saturated solution to approximately 0° C. within 0-30 min

Slow cooling: slowly cooling of a hot saturated solution to room temperature within 30 min to 4 hours.

Crystallization from mixed solvents: twice the amount of the solvent that shows lower solubility.

TABLE 4

| | | Form II |
|---|---|---|
| Solvent | Method | % (FTIR) |
| Acetone + H₂O | Fast cooling | >=70 |
| 1-BuOH + H₂O | Fast cooling | >=70 |
| 1-BuOH + H₂O | Slow cooling | >=70 |
| Dioxane | Fast cooling | >=70 |
| DMF + H₂O | Fast cooling | >=70 |
| ETOH + H₂O | Fast cooling | >=70 |
| ETOH + H₂O | Slow cooling | >=70 |
| Nitromethane + H₂O | Fast cooling | >=70 |
| Nitromethane + H₂O | Slow cooling | >=70 |
| 1-Propanol + H₂O | Fast cooling | >=70 |
| 1-Propanol + H₂O | Slow cooling | >=70 |
| Xylene | Fast cooling | >=70 |
| Xylene + acetone | Fast cooling | >=70 |
| Xylene + acetone | Slow cooling | >=70 |

### Example 9

### Preparation of Essentially Pure Polymorphic Form II of Aprepitant

0.4 g Aprepitant polymorph form III is suspended in 10 ml cis decahydronaphthalene and heated to about 120° C. in an oil bath for about 5 minutes. The resulting crystalline form is then isolated and dried in vacuum at room temperature.

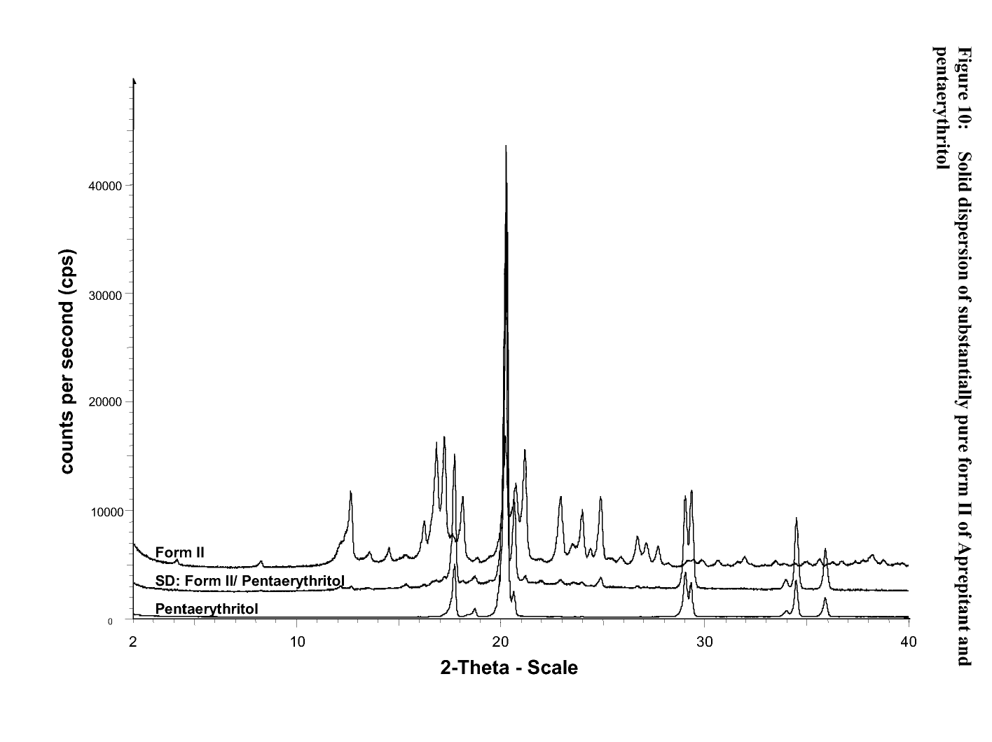

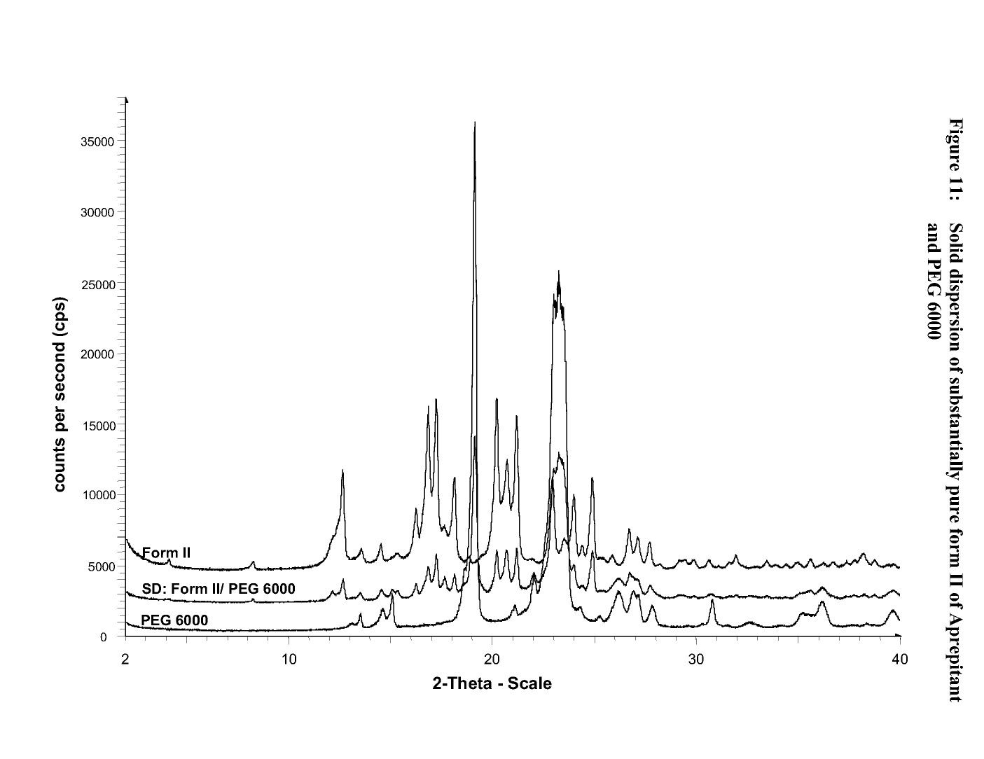

The XRPD pattern of the product is shown in FIG. 4 and the FT-IR spectrum is shown in FIG. 10 and curve A in the overlaid FT-IR spectra of FIG. 11. The crystalline form of the product is identified as essentially pure form II.

### Example 10

### Solid dispersion of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine form II

100 mg of PEG 6000 and 100 mg of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are mixed and the mixture is dissolved in acetone (approximately 10 ml). The mixture is concentrated at about 45° C. in vacuo to yield a residue.

Powder XRD shows the presence of crystalline form II of Aprepitant.

In a similar manner to example 4 solid dispersions of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine form II are obtained by dissolving carrier and 2-(R)-(1-(R)-(3,5)-bis(trifluoromethyl)-phenyl)ethoxy)-3-(S)-(4-fluoro) phenyl-4-(3-(5-oxo-1H,4H-1,2,4-triazolo)methylmorpholine using carriers and solvents as described in table 5.

TABLE 5

| Carrier | ratio Aprepitant:carrier | Solvent |
|---|---|---|
| pentaerythritol | 1:1 | Ethanol |
| PEG 6000 | 1:1 | Acetone |
| Sorbit | 1:1 | Ethanol/water |
| Mannit | 1:1 | Ethanol/water |
| Xylit | 1:1 | Ethanol |
| Dextrose | 1:1 | Methanol |
| Maltose | 1:1 | Methanol |
| Sucrose | 1:2 | Methanol |
| HPMCP | 1:1 | acetone/methanol |

### Example 11

Methanol solvate of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine

200 mg (0.2004 g) of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine are dissolved in 4.5 ml of methanol. The solution is then cooled to −15° C. and kept at this temperature for 24 hours. The needles are filtered to yield 0.1256 g of the title compound.

TABLE 7
Characteristic peaks of the powder pattern of
the methanol-solvate of Aprepitant

| 2-Theta (°) | rel Intentsity (%) |
|---|---|
| 9.72 | 9.6 |
| 10.53 | 100 |
| 14.08 | 20.4 |
| 21.21 | 20.8 |
| 22.26 | 6.6 |

1. Polymorphic form III of Aprepitant characterized by an X-ray powder diffraction pattern with peaks at 6.8±0.2°, 7.4±0.2°, 11.9±0.2°, 12.6±0.2°, 17.1±0.2°, 18.3±0.2°, 18.7±0.2° 19.3±0.2°, 19.7±0.2°, 20.2±0.2°, 20.6±0.2° and 21.0±0.2° degrees two theta.

2. Form III of Aprepitant of claim 1 characterized by an X-ray powder diffraction pattern substantially in accordance with FIG. 1.

3. Form III of Aprepitant of claim 1 characterized by an infrared spectrum substantially in accordance with FIG. 8.

4. A method of preparing form III of Aprepitant comprising the steps of:
a) dissolving 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine in an alicyclic ether or an aliphatic diether- to form a solution;
b) contacting the solution with C₅-C₁₀ aliphatic or alicyclic hydrocarbon to form a precipitate; and
c) isolating the precipitate, which is the form III of Aprepitant.

5. A method of claim 4 wherein the alicyclic ether in step a) comprises tetrahydrofuran.

6. A method of claim 4 wherein the aliphatic diether in step a) comprises at least one of dioxan or dimethoxyethan.

7. A method of claim 4 wherein the hydrocarbon in step b) comprises at least one of n-hexane, n-heptane or cyclohexane.

8. A method of claim 4 wherein a ratio of solvent and antisolvent in the precipitation step is between 1:9 and 1:30.

9. Method for the preparation of Aprepitant form II in essentially pure form containing no more than 5% of other crystalline forms of Aprepitant comprising converting a form III of Aprepitant to the form II Aprepitant in essentially pure form containing no more than 5% of other crystalline forms of Aprepitant.

10. A method of preparing Aprepitant form II in essentially pure form containing no more than 5% of other crystalline forms of Aprepitant according to claim 9, wherein the form III of Aprepitant is converted to the form II Aprepitant by heating a slurry of the form III Aprepitant to about 120° C. to form Aprepitant form II in essentially pure form containing no more than 5% of other crystalline forms.

11. The method of claim 10, wherein form III is a slurry in an alkane with a boiling range between 100-140° C.

12. The method of claim 11, wherein the alkane comprises at least one of petroleum benzene or decalin.

13. A pharmaceutical composition comprising form III of Aprepitant.

14. A pharmaceutical composition according to claim 13, further comprising one or more suitable excipients and additives.

15. A pharmaceutical composition according to claim 13, wherein the composition is in the form of a tablet, a capsule, a pill, a granule, or a powder for oral suspension.

16. A pharmaceutical composition comprising essentially pure form II of Aprepitant containing no more than 5% of other crystalline forms of Aprepitant.

17. A pharmaceutical composition according to claim 16, further comprising one or more suitable excipients and additives.

18. A pharmaceutical composition according to claim 16, wherein the composition is in the form of a tablet, a capsule, a pill, a granule, or a powder for oral suspension.

19. Aprepitant methanol solvate characterized by a X-ray powder diffraction pattern with peaks at 9.7±0.2°, 10.5±0.2°, 14.1±0.2°, 21.2±0.2° and 22.3±0.2°.

20. Aprepitant methanol solvate of claim 19 characterized by a X-ray powder diffraction pattern substantially in accordance with FIG. 2.

21. Aprepitant methanol solvate of claim 9 characterized by a DSC scan as shown in FIG. 24.

22. A process for the preparation of substantially pure crystalline form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine containing less than about 40% of form I comprising the steps of
a) providing a solution of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine in a solvent
b) high supersaturating the solution by mixing the solution with at least 2 volumes of an antisolvent and optionally adding seeds of form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine;
crystallizing form II from the solution to form precipitated crystals of form II;
d) isolating the precipitated crystals from the solution; and
e) drying the precipitated crystals.

23. A process according to claim 22, wherein the solvent comprises at least one of a ketone, a C₁-C₆ alcohol, a halogenated hydrocarbon, an ether or an ester.

24. A process according to claim 22, wherein the solvent comprises at least one of is acetone, methylethylketone, methylethylketone, 1-propanol, 2-propanol, chloroform, dichloromethane, dioxane, tetrahydrofuran, or ethyl acetate.

25. A process according to claim 22, wherein the antisolvent comprises at least one of water or an aliphatic hydrocarbon.

26. A process for the preparation of substantially pure form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine containing less than about 40% of form I comprising the steps of
a) providing a solution of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine in a solvent or solvent mixtures;
b) optionally evaporating some of the solvent to obtain a saturated solution;
c) optionally adding seeds of form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methyl morpholine to the solution;
d) crystallisation of a mixture of form II and form I by cooling of the solution
e) isolation of the precipitated crystals
f) drying of the precipitated crystals

27. A process according to claim 26, wherein the solvent comprises at least one selected from dioxane, tetrahydrofuran, toluene, xylene, 1-butanol or 2-propanol.

28. A process according to claim 26, wherein the solvent mixture comprises a mixture of 1 volume of a solvent selected from acetone, N,N-dimethylformamide, methanol, ethanol, 1-propanol, 2-propanol and at least at about 2 volumes of water.

29. A solid dispersion of substantially pure form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine containing less than about 40% of form I in a suitable carrier.

30. A solid dispersion according to claim 29, wherein the carrier comprises at least one selected from the group consisting of polymers, sugars, sugar alcohols, organic acids and cellulose derivatives.

31. A solid dispersion according to claim 30, wherein the carrier comprises at least one selected from PEG 6000, sorbitol, mannitol, xylitol, dextrose, maltose, sucrose or hydroxypropyl methylcellulose phthalate.

32. A process for the preparation of a solid dispersion according to claim 29, comprising the removal of a solvent or solvent mixture from a solution of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo) methylmorpholine and a carrier.

33. A process according to claim 32, wherein the solvent or solvent mixture is removed by lyophilization, evaporation or spray drying.

34. A process according to claim 32, wherein the solvent or solvent mixture comprises at least one selected from alcohols, ketones, mixtures of water and alcohols, and mixtures of ketones and alcohols.

35. A process according to claim 32, wherein the solvent or solvent mixture comprises at least one selected from ethanol, methanol, acetone, a mixture of ethanol and water, and a mixture of acetone and methanol.

36. A process to according to claim 32, wherein the carrier comprises at least one selected from pentaerythritol, polyethyleneglycol, sorbitol, mannitol, xylitol, dextrose, maltose, sucrose or hydroxypropyl methylcellulose phthalate.

37. A pharmaceutical composition comprising a solid dispersion of mixtures of substantially pure Aprepitant form II containing less than about 40% of form I in a suitable carrier.

38. A pharmaceutical composition according to claim 37, further comprising one or more suitable excipients and additives.

39. A pharmaceutical composition according to claim 37, wherein the composition is in the form of a tablet, a capsule, a pill, a granule, or a powder for oral suspension.

40. A pharmaceutical composition comprising substantially pure form II of Aprepitant containing less than about 40% of form I or a solid dispersion thereof characterised by that the substantially pure form II of Aprepitant is prepared according to a process comprising:
a) providing a solution of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine in a solvent;
b) high supersaturating the solution by mixing the solution with at least 2 volumes of an antisolvent and optionally adding seeds of form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine;
c) crystallizing form II from the solution to form precipitated crystals of form II;
d) isolating the precipitated crystals from the solution; and
e) drying the precipitated crystals.

41. A pharmaceutical composition according to claim 40, further comprising one or more suitable excipients and additives.

42. A pharmaceutical composition according to claim 40, wherein the composition is in the form of a tablet, a capsule, a pill, a granule, or a powder for oral suspension.

43. Stable substantially pure form II of 2-(R)-(1-(R)-(3,5)-bis(trifluoromethyl)-phenyl)ethoxy)-3-(S)-(4-fluoro)phenyl-4-(3-(5-oxo-1H,4H-1,2,4-triazolo)methylmorpholine containing less than about 40% of form I.
